(19) United States
(12) Patent Application Publication
(10) Pub. No.: US 2002/0071879 A1
(43) Pub. Date: Jun. 13, 2002

(54) Method and device for treating hemorrhoids

(76) Inventor: Winsor Eveland, Englewood, FL (US)

Correspondence Address: LAURA G. BARROW ESQ., P. O. BOX 215, ESTERO, FL, 33928-0215, US

(21) Appl. No.: 09/732,518

(22) Filed: Dec. 7, 2000

(51) Int. Cl. A61K 35/78; A61N 1/00

(52) U.S. Cl. 424/768; 607/1

(57) ABSTRACT

A method and device for treating hemorrhoids is described and illustrated herein comprising, in part, the application of external heat to the affected rectal area of a person suffering from hemorrhoids.

### BACKGROUND AND SUMMARY OF THE INVENTION

Hemorrhoids are caused by the swelling and thrombosis of a large plexus of veins in the anal canal followed by edema. Conditions that frequently cause the formation of hemorrhoids include chronic constipation, irregularity in bowel movements, poor diet, and pregnancy-induced interference with the venous return flow due to fetal pressures against the pelvic area. Hemorrhoids can be quite painful and on occasion may be accompanied by cracks in the anal cavity, resulting in bleeding and infection.

Current treatments for hemorrhoids include sitz baths three to four times daily in water as hot as the patient can tolerate comfortably. Patients often consume bulk stool formers to aid in bowel movements while acute hemorrhoids often require surgical intervention.

The present invention is directed to a method of treating external hemorrhoids by applying externally to the rectal area a stream of hot air at least once a day until shrinkage of the hemorrhoids results. Certain aspects of the present invention include a hot air blower specifically designed for applying a stream hot air to the rectal area with greater ease.

### BRIEF DESCRIPTION OF THE FIGURES

FIG. 1 is a side view of one embodiment of the hot air blower used in the present inventive method for treating hemorrhoids.

FIG. 2 is a front view of the nozzle portion of the hot air blower taken along lines 2-2 of FIG. 1.

FIGS. 3-4 illustrate the application of heat to the hemorrhoidal area.

### DETAILED DESCRIPTION OF THE PREFERRED EMBODIMENTS

The present invention, in certain aspects, is directed to a method of treating hemorrhoids comprising applying, at least once daily, and more preferably two to three times daily, a stream of hot air externally to the rectal area until shrinkage of the hemorrhoids occurs. While the inventive method is primarily effective for treating external hemorrhoids, it had been discovered that internal hemorrhoids and fissures slowly disappear subsequent to the successful treatment of the external hemorrhoidal condition.

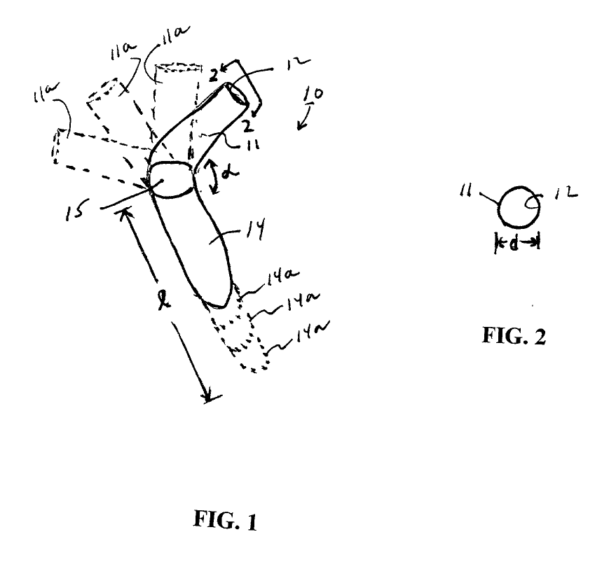

FIG. 1 illustrates a hot air blower (10) that may be employed in the present invention. Specifically, as shown in the figures, the hot air blower (10) comprises a nozzle portion (11) having an open end (12) through which a stream (13) of hot air is expelled. Preferably, the diameter (d) of the open end of the nozzle portion is 1.5 inches or less, preferably about 1 to 1.5 inches, in order to concentrate the stream of hot air to the critical area without affecting the surrounding body areas (i.e. lower buttocks, thighs, genitals). At the other end of the hot air blower is a handle (14) which is grasped during treatment. An elongated handle is desirable for self-treatment by the hemorrhoid sufferer, in particular for those persons who have large frames and/or are overweight and thus may have difficulty positioning the hot air blower to the desired area. For example, large people may need a longer handle to maneuver the hot air blower in order to apply heat to the critical rectal area. The preferred length (1) of the elongated handle is at least 7 inches; however, the handle length may also be adjustable, and preferably adjustable to a length (1) of at least 7 inches, as shown in phantom (11a) in FIG. 1.

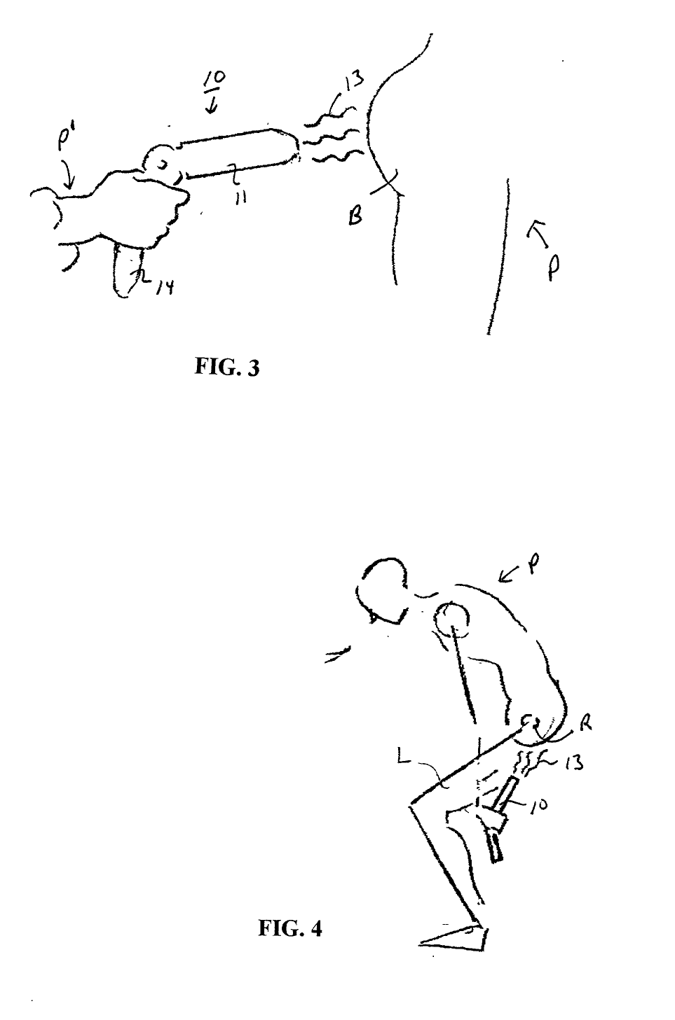

As shown in the figures, the hot air blower (10) of the present invention is much like a conventional hand-held blow dryer for drying hair. In fact, a hand-held hair dryer design could be employed. However, as discussed above, employment of a hot air blower having an elongated handle is desirable for those individuals who might have difficulty manipulating a conventionally-sized blow dryer for accurate application of the hot air stream to the rectal area. The nozzle portion (11) of the hot air blower may be fixed or it may be adjustable. Specifically, the nozzle portion (11) may be pivotally secured at one end (15) to the handle (14) such that the nozzle portion is adjustable to varying angles (α) relative to the handle, as shown in phantom (14a) in FIG. 1. The hot air blower (10) may be powered by any conventional power source, preferably by a 120 volt or 220 volt power source that heats the coil (not shown) within the hot air blower to a temperature not to exceed 140° F. (60° C.). As discussed in part above, the nozzle portion of the hot air blower is positioned near the affected rectal area (R), preferably about 1 to 2 inches away from the rectal area so that the nozzle is not touching the rectal area or buttocks (B) of the person (P) suffering from hemorrhoids. For self-treatment, the hemorrhoid sufferer may position the nozzle portion (11) of the hot air blower (10) between his/her legs (L) anteriorly, as shown in FIG. 4, to direct the stream (13) of hot air to the critical area. Alternatively, the hemorrhoid sufferer may approach the hemorrhoid affected area from his/her posterior side to direct the stream of hot air thereto, or a second person (P¹) may apply the heat to the hemorrhoids sufferer's affected area, as shown in FIG. 3, for example. A steady stream of hot air is then applied to the affected rectal area for at least 15 seconds, but more preferably for as long as the user can tolerate the heat. Such external hot air applications should be made at least once daily, and more preferably two to three times daily, for a sufficient number of days until the sufferer's hemorrhoids disappear or, at a minimum, hemorrhoidal swelling has significantly decreased. Typical treatment periods range from 30 to 90 days. To minimize recurrences or “flare-ups,” periodic applications of hot air should be made, preferably every 7-10 days.

In order to aid in the treatment of the hemorrhoidal condition, the sufferer may ingest flax seed, preferably ground flax seed, at least once daily. A preferred oral dose is 6 grams of flax seed administered daily. The purpose of the flax seed is to soften the stool, thereby aiding in preventing the recurrence of external hemorrhoids as well as possibly aiding in the reduction of internal hemorrhoids and fissures. Such administration of flax seed provides the sufferer a large dose of omega-3 and omega-6 fatty acids and thus, may aid in the reconstruction and strengthening of damaged or weakened transitional epithelial tissue around the anal orifice.

It will be appreciated by those of ordinary skill in the art that treatment durations will vary depending upon the patient and the severity of the hemorrhoidal condition. It will further be recognized that variations in size and construction of the hot air blower may be made without departing from the scope and spirit of the present invention. Also, various combinations and sub combinations of the features of the hot air blower component of the present invention may be made, as well, thereby resulting in a variety of different embodiments of the hot air blower without departing from the scope and spirit of the present invention.

### EXAMPLE 1

A 62-year-old male suffering from external hemorrhoids positioned a hot air blower approximately 1 to 2 inches from the rectal opening and subsequently applied a stream of hot air thereto for about 10-20 seconds twice daily. The treatment was repeated 10 daily for 30 days, after which time the hemorrhoids disappeared.

I claim:

1. A method of treating hemorrhoids comprising:
externally applying, at least once daily, a stream of hot air to a hemorrhoid affected rectal area of a person suffering therefrom;
wherein said stream of hot air is applied by said person using a hot air blower, said blower comprising:
(a) a nozzle portion, said nozzle portion having an open end through which said stream of hot air is expelled; and
(b) a handle secured to an opposite end of said nozzle portion.

2. The method of claim 1, wherein said stream of hot air is applied continuously to said affected rectal area for at least 15 seconds.

3. The method of claim 1, wherein said stream of hot air has a temperature of 140° F. or less.

4. The method of claim 1, wherein said open end of said nozzle portion has a diameter of 1.5 inches or less.

5. The method of claim 4, wherein said stream of hot air is applied continuously to said affected rectal area for at least 15 seconds.

6. The method of claim 5, wherein said stream of hot air has a temperature of 140° F. or less.

7. The method of claim 1, further including orally administering a therapeutically effective amount of flax seed to said person.

8. The method of claim 7, wherein said effective amount is 6 grams administered daily.

9. The method of claim 1, wherein said nozzle portion is pivotally secured to said handle such that said nozzle portion may be adjustable at varying angles relative to said handle.

10. The method of claim 9, wherein said stream of hot air is applied continuously to said affected rectal area for at least 15 seconds.

11 The method of claim 9, wherein said stream of hot air has a temperature of 140° F. or less.

12. The method of claim 9, wherein said open end of said nozzle portion has a diameter of 1.5 inches or less.

13. The method of claim 12, wherein said stream of hot air is applied continuously to said affected rectal area for at least 15 seconds.

14. The method of claim 13, wherein said stream of hot air has a temperature of 140° F. or less.

15. The method of claim 9, further including orally administering a therapeutically effective amount of flax seed to said person.

16. The method of claim 15, wherein said effective amount is 6 grams administered daily.

17. A method of treating hemorrhoids comprising:
externally applying, at least once daily, a stream of hot air to a hemorrhoid affected rectal area of a person suffering therefrom;
wherein said stream of hot air is applied by said person using a hot air blower, said blower comprising:
(a) a nozzle portion, said nozzle portion having an open end through which said stream of hot air is expelled; and
(b) an elongated handle secured to an opposite end of said nozzle portion, said handle having a length of at least 7 inches.

18. The method of claim 17, wherein said stream of hot air is applied continuously to said affected rectal area for at least 15 seconds.

19. The method of claim 17, wherein said stream of hot air has a temperature of 140° F. or less.

20. The method of claim 17, wherein said open end of said nozzle portion has a diameter of 1.5 inches or less.

21. The method of claim 20, wherein said stream of hot air is applied continuously to said affected rectal area for at least 15 seconds.

22. The method of claim 21, wherein said stream of hot air has a temperature of 140° F. or less.

23. The method of claim 17, further including orally administering a therapeutically effective amount of flax seed to said person.

24. The method of claim 23, wherein said effective amount is 6 grams administered daily.

25. The method of claim 17, wherein said nozzle portion is pivotally secured to said handle such that said nozzle portion may be adjustable at varying angles relative to said handle.

26. The method of claim 25, wherein said stream of hot air is applied continuously to said affected rectal area for at least 15 seconds.

27. The method of claim 25, wherein said stream of hot air has a temperature of 140° F. or less.

28. The method of claim 25, wherein said open end of said nozzle portion has a diameter of 1.5 inches or less.

29. The method of claim 28, wherein said stream of hot air is applied continuously to said affected rectal area for at least 15 seconds.

30. The method of claim 29, wherein said stream of hot air has a temperature of 140° F. or less.

31. The method of claim 25, further including orally administering a therapeutically effective amount of flax seed to said person.

32. The method of claim 31, wherein said effective amount is 6 grams administered daily.

33. A method of treating hemorrhoids comprising:
externally applying, at least once daily, a stream of hot air to a hemorrhoid affected rectal area of a person suffering therefrom;
wherein said stream of hot air is applied by said person using a hot air blower, said blower comprising:
(a) a nozzle portion, said nozzle portion having an open end through which said stream of hot air is expelled; and
(c) an elongated handle secured to an opposite end of said nozzle portion, said handle further having an adjustable length.

34. The method of claim 33, wherein said stream of hot air is applied continuously to said affected rectal area for at least 15 seconds.

35. The method of claim 33, wherein said stream of hot air has a temperature of 140° F. or less.

36. The method of claim 33, wherein said open end of said nozzle portion has a diameter of 1.5 inches or less.

37. The method of claim 36, wherein said stream of hot air is applied continuously to said affected rectal area for at least 15 seconds.

38. The method of claim 37, wherein said stream of hot air has a temperature of 140° F. or less.

39. The method of claim 33, further including orally administering a therapeutically effective amount of flax seed to said person.

40. The method of claim 39, wherein said effective amount is 6 grams administered daily.

41. The method of claim 38, wherein said handle length is adjustable to at least 7 inches.

42. The method of claim 41, wherein said stream of hot air is applied continuously to said affected rectal area for at least 15 seconds.

43. The method of claim 41, wherein said stream of hot air has a temperature of 140° F. or less.

44. The method of claim 41, wherein said open end of said nozzle portion has a diameter of 1.5 inches or less.

45. The method of claim 44, wherein said stream of hot air is applied continuously to said affected rectal area for at least 15 seconds.

46. The method of claim 45, wherein said stream of hot air has a temperature of 140° F. or less.

47. The method of claim 41, further including orally administering a therapeutically effective amount of flax seed to said person.

48. The method of claim 47, wherein said effective amount is 6 grams administered daily.

50. The method of claim 33, wherein said nozzle portion is pivotally secured to said handle such that said nozzle portion may be adjustable at varying degree angles relative to said handle.

51. The method of claim 50, wherein said stream of hot air is applied continuously to said affected rectal area for at least 15 seconds.

52. The method of claim 50, wherein said stream of hot air has a temperature of 140° F. or less.

53. The method of claim 50, wherein said open end of said nozzle portion has a diameter of 1.5 inches or less.

54. The method of claim 53, wherein said stream of hot air is applied continuously to said affected rectal area for at least 15 seconds.

55. The method of claim 54, wherein said stream of hot air has a temperature of 140° F. or less.

56. The method of claim 54, further including orally administering a therapeutically effective amount of flax seed to said person.

57. The method of claim 56, wherein said effective amount is 6 grams administered daily.

58. The method of claim 50, wherein said wherein said handle length is adjustable to at least 7 inches.

59. The method of claim 58, wherein said stream of hot air is applied continuously to said affected rectal area for at least 15 seconds.

60. The method of claim 58, wherein said stream of hot air has a temperature of 140° F. or less.

61. The method of claim 58, wherein said open end of said nozzle portion has a diameter of 1.5 inches or less.

62. The method of claim 61, wherein said stream of hot air is applied continuously to said affected rectal area for at least 15 seconds.

63. The method of claim 62, wherein said stream of hot air has a temperature of 140° F. or less.

64. The method of claim 58, further including orally administering a therapeutically effective amount of flax seed to said person.

65. The method of claim 64, wherein said effective amount is 6 grams administered daily.

66. A therapeutic hemorrhoid treatment kit comprising:
(a) flax seed for oral administration; and
(b) a hot air blower designed for externally applying a stream of hot air to a rectal area of a person suffering from hemorrhoids, said hot air blower further including (i) a nozzle portion, said nozzle portion having an open end through which said hot air is expelled and (ii) a handle secured to an opposite end of said nozzle portion.

67. The kit of claim 66, wherein said open end of said nozzle portion of said blower has a diameter of 1.5 inches or less.

68. The kit of claim 67, wherein said nozzle portion is pivotally secured to said handle such that said nozzle portion may be adjustable at varying angles relative to said handle.

69. The kit of claim 66, wherein said handle has a length of at least 7 inches.

70. The kit of claim 69, wherein said nozzle portion is pivotally secured to said handle such that said nozzle portion may be adjustable at varying degree angles relative to said handle.

71. The kit of claim 70, wherein said open end of said nozzle portion of said blower has a diameter of 1.5 inches or less.

72. The kit of claim 66, wherein said handle has an adjustable length.

73. The kit of claim 72, wherein said nozzle portion is pivotally secured to said handle such that said nozzle portion may be adjustable at varying angles relative to said handle.

74. The kit of claim 73, wherein said open end of said nozzle portion of said blower has a diameter of 1.5 inches or less.

75. The kit of claim 74, wherein said handle length is adjustable to at least 7 inches.
